(19) United States
(12) Patent Application Publication
(10) Pub. No.: US 2009/0227712 A1
(43) Pub. Date: Sep. 10, 2009

(54) Triazinyl flavonate brighteners

(76) Inventors: Heinz Giesecke, Odenthal (DE); Reiner Gottschalk, Köln (DE); Bernhard Hunke, Eitorf (DE); Dirk Pfuetzenreuter, Burscheid (DE)

Correspondence Address: CANTOR COLBURN, LLP, 20 Church Street, 22nd Floor, Hartford, CT, 06103, US

(21) Appl. No.: 11/630,060

(22) PCT Filed: Jun. 15, 2005
(86) PCT No.: PCT/EP05/06421

§ 371 Date: Sep. 25, 2007

(30) Foreign Application Priority Data
Jun. 28, 2004 (DE) 102004031101.3

(51) Int. Cl. C08K 5/3492; C07D 251/68

(52) U.S. Cl. 524/100; 544/193.2; 106/503

(57) ABSTRACT

The invention relates to compounds of formula (I) wherein R¹, R², R³ and R⁴ independently represent OR³ or NR⁶R⁷; R⁵, R⁶ and R⁷ independently represent hydrogen, substituted or unsubstituted alkyl, especially C₁-C₄ alkyl, or substituted or unsubstituted aryl, especially C₆-C₁₀ aryl; R⁶, R⁷ can also form an aliphatic or aromatic ring, together with the N atom to which they are bonded and optionally other heteroatoms; M represents hydrogen, an equivalent of a monovalent or bivalent metal ion, especially from the group of alkali metals or alkaline earth metals, or of an optionally organically substituted ammonium ion; and B¹ represents a bivalent binding link.

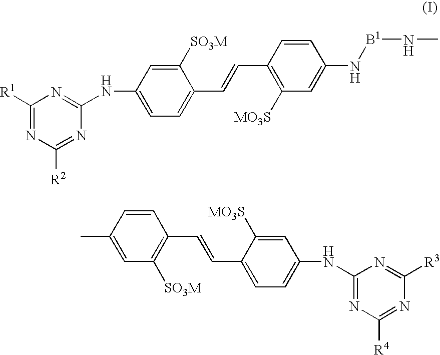

The invention relates to new triazinyl-flavonate brighteners, process for their production, preparations containing them, and their use, especially as brighteners for paper coatings.

The addition of optical brighteners to coating colorants is common in the production of coated papers, so that the optical brightener in the finished coated paper is also found in the pigment layer applied to the paper. Coated papers are particularly suitable for the production of high-quality printed matter. In addition to good printability properties, their quality is therefore mostly evaluated according to optical properties such as gloss, smoothness, and whiteness. There is a continuous trend toward coated papers with high whiteness, and therefore a desire for the most effective possible optical brighteners as coating components.

Triazinyl-flavonate brighteners are used worldwide to a large extent to whiten paper and textiles in the paper, textile, and detergent industries. This class of brighteners is generally constructed from a 4,4′-diaminostilbene-2,2′-disulfonic-acid middle piece and two triazine residues that additionally contain four optionally further substituted amine and/or alkoxy or aryloxy groups. Triazinyl-flavonate brighteners of this structure with only a 4,4′-diaminostilbene-2,2′-disulfonic-acid core, hereafter referred to as single-core triazinyl-flavonate brighteners (with reference to the middle piece) are suitable to a large extent for providing the desired brightening effects in the areas of application mentioned, especially also in paper coating.

Appropriate single-core triazinyl-flavonate brighteners for paper coating have long been known, for example from EP-A-1 355 004, and are still capable of improvement with respect to their whiteness.

Based on the continued trend mentioned toward coated papers with high whiteness and the demand that therefore exists for the most effective possible optical brighteners, there is interest in providing optical brighteners with increased efficiency with respect to the known single-core triazinyl-flavonate brighteners.

Surprisingly, new compounds of formula I have now been found, which can be referred to in the above sense as two-core triazinyl-flavonate brighteners

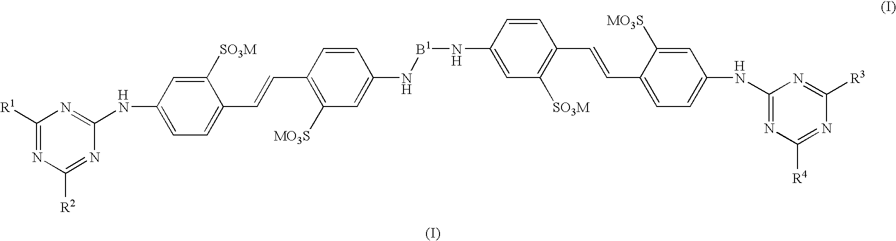

in which
R¹, R², R³, and R⁴ each, independently of one another, stand for OR⁵ or NR⁶R⁷, whereby
R⁵, R⁶, and R⁷ each, independently of one another stand for hydrogen, a substituted or unsubstituted alkyl, especially a C₁-C₄ alkyl or a substituted or unsubstituted aryl, especially a C₆-C₁₀ aryl, whereby
R⁶, R⁷ can also form an aliphatic or aromatic ring together with the N atom to which they are bonded and optionally additional heteroatoms,
M stands for hydrogen, an equivalent of a mono- or divalent metal ion, especially from the group of alkali or alkaline-earth metals, or an optionally organically substituted ammonium ion, and
B¹ stands for a bivalent bridge element.

Preferred compounds of formula I are those in which at least one of the groups R¹ or R² has the same meaning as at least one of the groups R³ or R⁴. Especially preferred is R¹═R³ and R²═R⁴. It is also especially preferred that groups R¹ to R⁴ are identical.

M preferably stands for hydrogen or alkali or alkaline-earth metals, especially lithium, sodium, potassium, or an equivalent of magnesium or calcium, as well as with particular preference ammonium and hydroxyalkyl-substituted, especially hydroxyl-substituted C₁-C₄ alkyl ammonium.

The bridge element B¹ preferably stands for a group of the formula

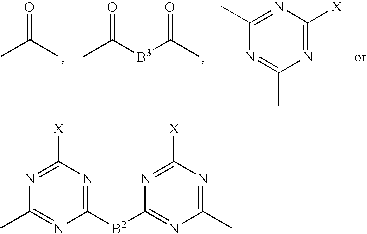

in which
B³ denotes a bivalent, aliphatic, or aromatic group, especially

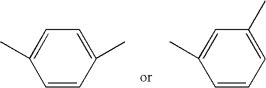

and
X stands for OR⁵ or NR⁶R⁷, whereby
R⁵, R⁶, and R⁷ each, independently of one another have the meanings given above and
B² denotes a bridge element bonded through oxygen atoms or nitrogen atoms to the triazine groups, preferably an aliphatic bridge element.

Of the preferred group mentioned, bridge elements carrying an X substituent in which X in particular is identical to at least one of the groups R¹ to R⁴ or stands for hydroxy are especially preferred.

Preferred groups R¹, R², R³, and R⁴ are the following: phenoxy, mono- or disulfonated phenoxy, phenylamino, mono- or disulfonated phenylamino, phenylamino substituted with C₁-C₃ alkyl, cyano, halogen, especially Cl or Br, COOR, CONH—R, NH—COR, SO₂NH—R, OR, also the groups morpholino, piperidino, pyrrolidino, —OC₁-C₄ alkyl, —NH—(C₁-C₄ alkyl), —N(C₁-C₄ alkyl)₂, —NH(C₂-C₄ alkylene)-OR, —N[(C₁-C₄-alkylene)-OR]₂, —NH(C₂-C₄ hydroxyalkyl), —N(C₂-C₄ hydroxyalkyl)₂, —NH(C₂-C₄ alkylene-O—C₂-C₄-alkylene-OR), an amino acid or amino-acid salt, or an amino-acid amide, from whose basic amino group a hydrogen atom is removed, —N(CH₃)(CH₂CH₂OH), —NH₂, —OCH₂CH₂SO₃M, —NH—CH₂CH₂SO₃M, —N(CH₂CH₂SO₃M)₂, or —N(CH₂CH₂OH)CH₂CH₂CONH₂, whereby R═H or C₁-C₃ alkyl and M has the above-mentioned meaning.

Among these, particularly preferred are the groups —NH₂, —NH—CH₃, —NH—C₂H₅, —N(CH₃)₂, —N(C₂H₅)₂, —NH—C₂-C₄ hydroxyalkyl, especially —NH—CH₂CH₂OH, —N(C₂-C₄ hydroxy-alkyl)₂, —NH—CH₂CH₂SO₃M, —NH—CH₂—CH₂—O—CH₂—CH₂—OH, —OCH₃, —OCH(CH₃)₂, —O—CH₂—CH₂—O—CH₃, —N(CH₂—CH₂—OH)₂, —N(CH₂—CHOH—CH₃)₂, morpholino, —N(CH₂—CH₂—OH)CH₂—CH₂—CONH₂, as well as groups of the formula

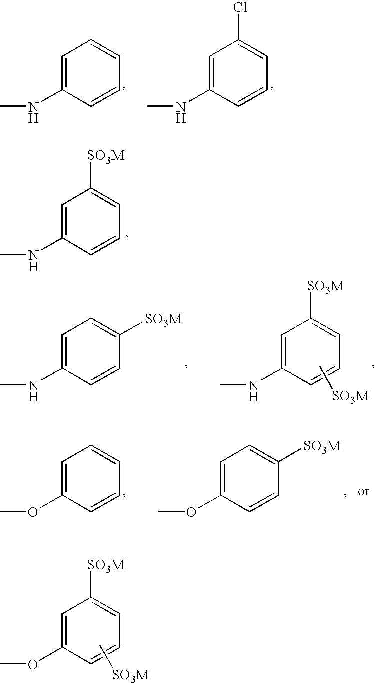

in which
M has the above-mentioned meaning.

Especially preferably, groups R¹ to R⁴, independently of one another, stand for

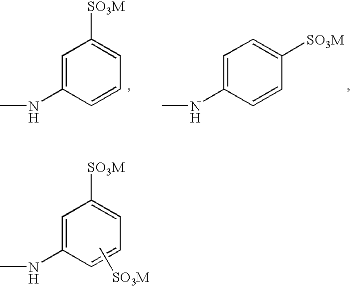

—NH—CH₂CH₂OH, —N(CH₂—CH₂—OH)₂, —N(CH₂—CHOH—CH₃)₂, aniline, or morpholino.

Especially preferred compounds are those of formula IA

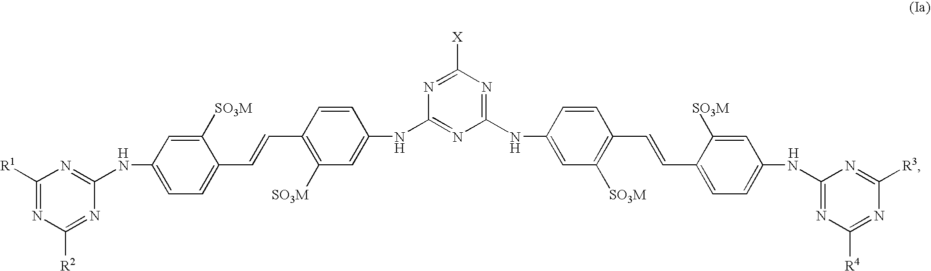

in which
R¹ and R³, independently of each other, stand for a group of formula

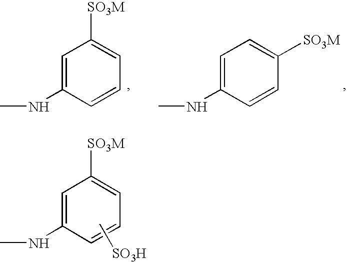

in which
M has the above meaning,
R² and R⁴ independently of each other, stand for —NHCH₂—CH₂OH, —N(CH₂CH₂OH)₂,

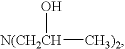

anilino or morpholino, and
X stands for OH or for —NHCH₂CH₂OH, —N(CH₂CH₂OH)₂,

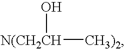

aniline, or morpholino.

Another object of the present invention is a process for producing triazinyl-flavonate brighteners according to the invention, characterized in that a compound of formula II

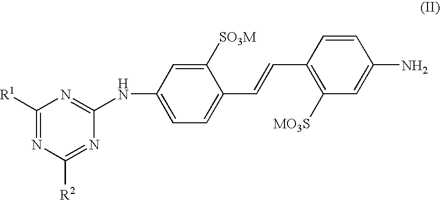

in which
R¹, R², and M have the above-mentioned meaning, is converted with a compound of formula (III)

Y—B¹-Z (III)

in which
Y and Z independently of each other, stand for leaving groups that can be replaced by the free amino group of the compounds of formula II and B¹ has the above-mentioned meaning, into a compound of formula IIIa

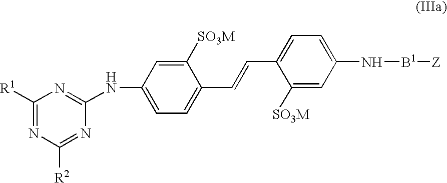

in which
R¹, R², B¹, and Z have the above-mentioned meaning,
   and compound IIIa is further converted with a compound of formula IV

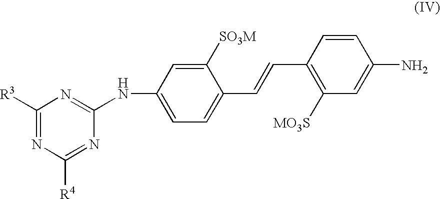

in which R³, R⁴, and M have the above-mentioned meaning.

Leaving groups Y and Z in formula III are preferably understood to mean molecule fragments that differ from each other or are also the same and are split off during a condensation reaction of a compound of formula II or IV with a compound of formula III as compounds Y—H or Z-H. Splitting off of the second leaving group is preferably less easy than that of the first, so that the most selective possible 1:1 conversion of the compound of formula II with the compound of formula III can occur, so that in the condensation product being for med essentially only one of the groups Y or Z is retained, this being particularly important when asymmetric compounds of formula I are to be produced, i.e., at least R¹≠R³ and/or R²≠R⁴.

Y and Z, independently of each other, preferably stand for a halogen, especially fluorine, chlorine, or bromine, especially chlorine, alkoxy, especially C₁-C₄ alkoxy, or aryloxy, especially optionally substituted phenoxy.

Chlorocarbonic-acid esters, especially their C₁-C₄ alkyl esters or 2,4,6-trichlorotriazine are considered as preferred compounds of formula III.

Reaction of II with III preferably occurs at a temperature from 5 to 50° C. The preferred molar ratio of II to III is 0.85 to 1.15.

Reaction of the reaction product from II and III, i.e., IIIa, with compounds of formula IV preferably occurs at a temperature from 20 to 100° C. The mole ratio of II to IV is preferably 0.95 to 1.05.

Water or organic solvents fully or partially miscible with water or mixtures thereof can be considered as reaction media for the conversion of compounds of formula II-IV, for example acetone, methylethyl ketone, methylethyl-ketone/water mixtures, etc. The reactants can then be present either dissolved or as a suspension or emulsion or in mixed forms.

An aqueous reaction medium is particular preferred, which can optionally contain additional components, such as inorganic salts, emulsifiers, etc.

The pH value of the reaction mixture is preferably chosen for the individual reaction stages so that the correspondingly performed stage can occur as selectively as possible and at a high rate.

For a case in which the substance of formula III contains an additional leaving group apart from Y and Z, this can preferably be converted to the end product of formula I by subsequent reaction with an alcohol R⁵OH or an amine (R⁶, R⁷)NH, whereby R⁵, R⁶, and R⁷ have the above-mentioned meaning.

In a preferred variant of the invention, the process according to the invention is conducted using identical compounds of formula II and formula IV.

Compounds of formula II or IV can be prepared according to known processes in which cyanuric chloride, for example, preferably in a mole ratio of about 1:1, is converted in an arbitrary sequence with a compound of formula V

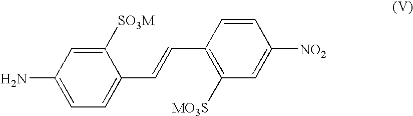

in which
M has the above-mentioned meaning
   and with compounds of formulas R¹H and R²H or R³H and R⁴H, in which R¹-R⁴ have the above-mentioned meaning and the nitro group of the compounds of formula VI formed

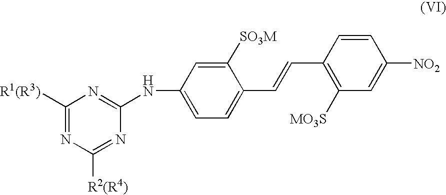

is then reduced to an amino group in a known way.

The brighteners of formula I according to the invention have distinctly improved white build-up behavior, especially when used in pigmented coating on paper, preferably in combination with other flavonate brighteners, with respect to the non-doubled brighteners. The desired brightening effect can therefore be achieved with a smaller amount of brightening agent.

The invention also concerns preparations containing at least one brightener of formula I according to the invention.

The preparations mentioned can contain other substances in addition to the triazinyl-flavonate brighteners according to the invention, for example, water, carrier substances, salts, and additives. They can also contain larger amounts of already known brighteners from the group of triazinyl-flavonate brighteners, those with one 4,4′-diaminostilbene-2,2′-disulfonic-acid group being preferred.

The new preparations generally contain the triazinyl-flavonate brighteners of formula I in an amount from 1 to 20 area percent with respect to total triazinyl-flavonate brighteners. This area percent is measured in high-pressure liquid chromatogram at a wavelength of 350 nm on an aqueous measurement solution of the preparation, whereby the amount of preparation expressed in grams to be weighed in per 100 ml of measurement solution should amount to 50 when multiplied by its E1/1 value. Aqueous brightener preparations are ordinarily characterized by the so-called E1/1 value. For this purpose, the extinction of a highly dilute solution of the preparation is determined according to the usual process known to one skilled in the art of UV/Vis spectroscopy in a 1-cm cell at a specified wavelength. This wavelength corresponds to the long-wave absorption maximum of the corresponding brightener molecule. It is about 350 nm in flavonate brighteners. The E1/1 value then corresponds to the fictitious extinction value extrapolated to a 1% solution of the sample being determined.

Preferred measurement conditions for the high-pressure liquid chromatogram are: separation with a reverse phase (RP) column and aqueous eluents (for example, buffered with acetonitrile and an ion-pair reagent).

Preferred new preparations contain the triazinyl-flavonate brighteners of formula I in an amount from 1 to 15% with respect to total brighteners, especially 1 to 10 area %, 2 to 10 area %, preferably 2.5 to 10 area %, especially preferably 3.0 to 10 area %, as determined above. Other preferred lower limits are 3.5, 4.0, 4.5, and 5.0 area %.

In addition, the preparations can be present as aqueous preparations, especially as solutions, but they also have solid forms and can be present, for example, as a powder or granulate.

Preferred preparations are aqueous and contain
5 to 50 wt % brightener, whereby which at least one of the brighteners corresponds to the compound of formula I according to the invention,
0 to 60 wt % carrier.

A brightener of formula VII is used preferably with the brightener to be used in the preparation

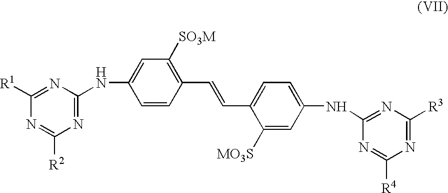

in which
R¹ to R⁴ and M have the above-mentioned meaning.

The sum of brighteners of formula I and VII of the preparation is preferably more than 70 wt %, especially more than 80 wt % with respect to total brightener content.

The preparation according to the invention especially preferably contains a mixture of brighteners containing at least one compound of formula I and at least one compound of formula VII. Most especially preferably, groups R¹ to R⁴ and M each have the same meaning in formulas I and VII.

The above information applies to the proportion of formula I in this mixture.

Generally, hydrophilic polymers with the capability of forming hydrogen bridge bonds are considered as carrier substances. Preferred carrier substances are polyvinyl alcohols, carboxymethyl celluloses, as well as polyethylene glycols with average molecular weights from 200 to 8000 g/mol, as well as any mixtures of these substances, whereby these polymers can optionally be modified. Preferred polyvinyl alcohols are those with a degree of hydrolysis >85%, and preferred carboxymethyl celluloses are those with a degree of substitution DS of >0.5. Especially preferred are polyethylene glycols with average molecular weights Mₙ from 200 to 8000 g/mol.

Native, derived or degraded starches, alginates, casein, proteins, polyacrylamides, hydroxyalkylcelluloses, and polyvinylpyrrolidone can also be considered.

Preparations according to the invention that contain the already known brighteners from the group of triazinyl-flavonate brighteners in addition to the new triazinyl-flavonate brighteners can be prepared, with respect to their brightener-active components by mixing the new triazinyl-flavonate brighteners as pure substances or in the form of solutions of the pure substances of appropriate concentration with the known triazinyl-flavonate brighteners.

As advantages of the preparation according to the invention, the new preparations are characterized during use in pigmented coating on paper during use at equal extinction (compared with triazinyl-flavonate brightener preparations that do not contain the new triazinyl-flavonate brighteners, but otherwise have the same composition) by improved white build-up behavior and higher maximum attainable whiteness, so that a greater brightening effect can be achieved in this way with identical use. As an alternative, if desired, an equally high brightening effect can be achieved with more limited use.

The invention also concerns coating masses brightened with the brighteners according to the invention or their preparations containing
water,
at least one white pigment,
at least one binder, especially latex binders, and
at least one brightener of formula I or a preparation containing the brightener of formula I.

The amount of binder, especially a latex binder (calculated as dry substance) is preferably 3 to 20 wt %, especially 5 to 15 wt %; independently of this, the amount of an optionally used synthetic cobinder different from it is 0.1 to 3 wt %, especially 0.5 to 1.5 wt %, and also, independently of it, the amount of brightener of formula I or the preparation containing it with respect to the brightener-active components, is 0.025 to 1 wt %, in each case with respect to the amount of white pigment.

The coating mass preferably also contains at least dispersant, especially in an amount from 0.05 to 1 wt % with respect to the white pigment in the coating mass. Polyacrylic acids and their corresponding salts are preferably considered as the dispersant. The water content of the coating mass is preferably 30 to 50 wt % with respect to the total amount of coating mass.

Calcium carbonate in natural or precipitated form, kaolin, talc, titanium dioxide, satin white, aluminum hydroxide, and barium sulfate are ordinarily used as white pigments, also in the form of mixtures.

All common latex formers that are used to produce paper coating masses can be considered as latex binders. As synthetic cobinders different from them, the coating masses contain, for example, carboxymethyl cellulose, hydroxyalkyl cellulose and/or polyvinyl alcohol, as well as synthetic thickeners based on acrylate.

### PREPARATION EXAMPLES

### Example 1

### Preparation of a Compound of Formula VIII

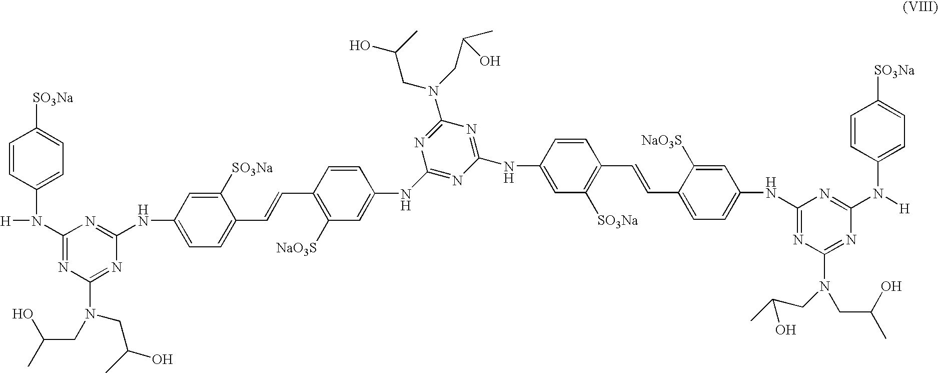

### Stage 1:

A solution of 0.53 mol sodium sulfanilate in 460 g water is added over about 1 hour to an agitated suspension 0.54 mol cyanuric chloride in 600 g water containing an emulsifier at 8° C., while the pH value is kept between 2.1 and 2.3 and the temperature below 25° C. by simultaneous addition of a 15% soda solution. After the addition is complete, agitation is carried out for about 40 minutes below 25° C. in the stated pH range. The total amount of soda solution required is about 187 g.

The pH value is then set to about 6.8 with 15% soda solution, and an aqueous solution of 0.52 mol of the sodium salt of 4-amino-4′-nitrostilbene-2,2′-disulfonic acid is added over about an hour, while the reaction mixture is simultaneously heated to about 35° C. and the pH value kept at 6.8 by adding 15% soda solution. After the addition completed, heating to 50° C. is carried out while still maintaining the pH value and continued under these conditions for 20 minutes. About 184 g of 15% soda solution is consumed.

0.68 mol of aqueous 84% diisopropanolamine solution is admitted to the reaction mixture at about 50° C., starting within 15 minutes, with simultaneous heating to 100° C. The pH value drops and is kept at 7.4 by adding 15% soda solution. It is agitated further at pH 7.4 and 100° C. for 3 hours.

An aqueous, salt-containing solution is obtained that contains about 0.5 mol of the compound of formula VI with

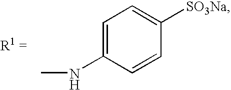

### and R²=—N(CH₂—CHOH—CH₃)₂.

### Stage 2:

About 380 g of 37% hydrochloric acid is added dropwise to an agitated suspension of about 7 mol iron powder (“for reduction” grade) in 800 g water of 90-100° C., during which a pH value of less than 1 is briefly set. 0.5 mol of the compound of formula VI with

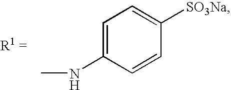

and R²=—N(CH₂—CHOH—CH₃)₂ in the form of the above-mentioned aqueous solution of stage 1 is then introduced over 1 hour. After the addition of stage 1 is completed, agitation is continued for 1.5 hours at 98-100° C., it is allowed to cool to 80° C. and filtration from the iron sludge is carried out.

The filtrate is cooled to 30° C. 1800 g of 15% soda solution is added dropwise at this temperature through which the pH rises to about 9. The precipitated basic iron carbonate is filtered away. The pH value of the filtrate is set at 2.0-2.2 by adding of 900 g 37% hydrochloric acid. 500 g sodium chloride is added, and it is agitated overnight.

The crystallized product is isolated with a suction filter and filtered for 20 hours until dry. 1400 g water is then added, and it is agitated so that thick suspension is formed. The pH value is set at 7-8 with 292 g 15% soda solution, and it is agitated to an almost clear solution and filtered. A solution is obtained that contains 0.34 mol of the compound of formula II with

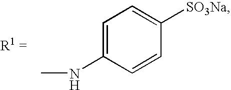

### and R²=—N(CH₂—CHOH—CH₃)₂.

### Stage 3:

A solution of 0.17 mol of the above-mentioned solution of stage 2 is added over about 30 minutes to an agitated suspension of 0.17 mol cyanuric chloride in 500 g containing an emulsifer at 8° C., while the pH value is kept between 4 and 4.5 by simultaneous addition of 15% soda solution, and the temperature is kept below 25° C. The total amount of soda solution required is about 60 g.

The pH value is then set to about 6.8 with 15% soda solution, and another 0.17 mol of the above-mentioned solution of stage 2 is added over about 1 hour, while the reaction mixture is simultaneously heated to 35° C. and the pH value is kept at 6.8 by adding a 15% soda solution. After the addition is complete, heating is carried out while still holding the pH value at 50° C., and agitation continued under these conditions for 20 minutes. About 60 g of 15% soda solution is consumed.

### Stage 4:

0.23 mol of an aqueous 84% diisopropanolamine solution is introduced into the reaction mixture at about 50° C., starting within 15 minutes, while heating is simultaneously carried out to 100° C. The pH value drops, and it is stopped at 7.4 by adding a 15% soda solution. After pH 7.4 is reached, agitation is continued for 3 hours at pH 7.4 and 100° C.

An aqueous, salt-containing solution is obtained, which contains about 0.17 mol of the compound of formula VIII

### Example 2

If 0.23 mol 90% diethanolamine solution is used in stage 4 of example 1 instead of 0.23 mol 84% diisopropanolamine solution and the procedure is otherwise followed as described in example 1, stage 4, an aqueous salt-containing solution is obtained that contains about 0.17 mol of formula IX.

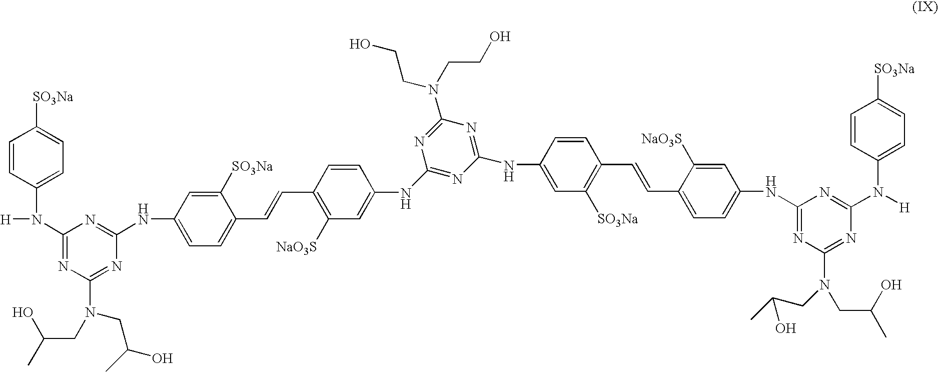

### Example 3

If 0.68 mol diethanolamine solution (90%) is used in stage 1 of example 1 instead of 0.68 mol 84% diisopropanolamine solution and the procedure is otherwise followed as described in example 1, stages 2 to 4, an aqueous salt-containing solution is obtained that contains about 0.15 mol of the compound of formula X.

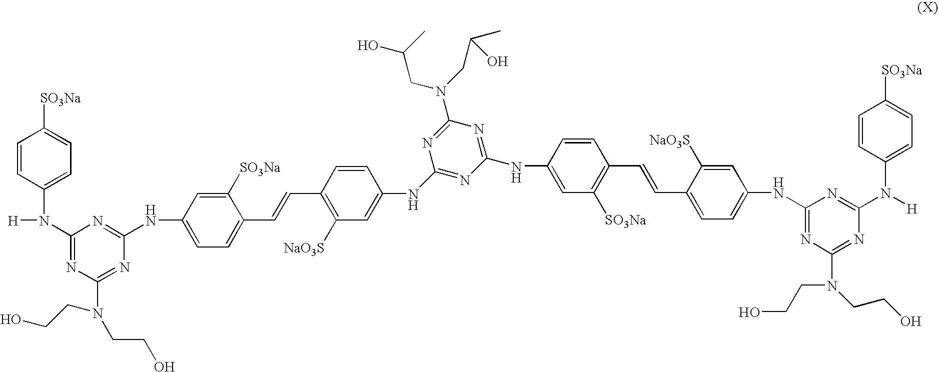

### Example 4

If 0.23 mol 90% diethanolamine solution is used in stage 4 of example 3 instead of 0.23 mol 84% diisopropanolamine solution and the procedure as described in example 1 is otherwise followed, an aqueous salt-containing solution is obtained that contains about 0.16 mol of the compound of formula XI.

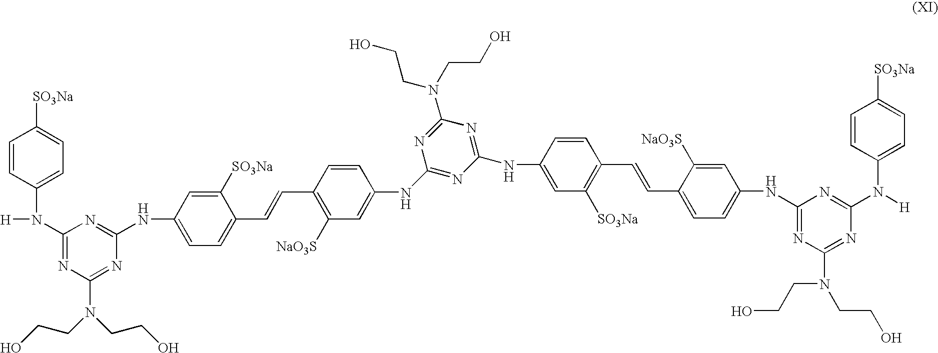

### Example 5

If in example 1, stage 3, 0.17 mol of the solution of stage 2 is no longer added at a pH 6.8 but instead of this an equimolar solution prepared according to example 3, stage 2, and the subsequent procedure of example 1, stage 4, is followed, an aqueous salt-containing solution is obtained that contains about 0.15 mol of the compound of formula XII.

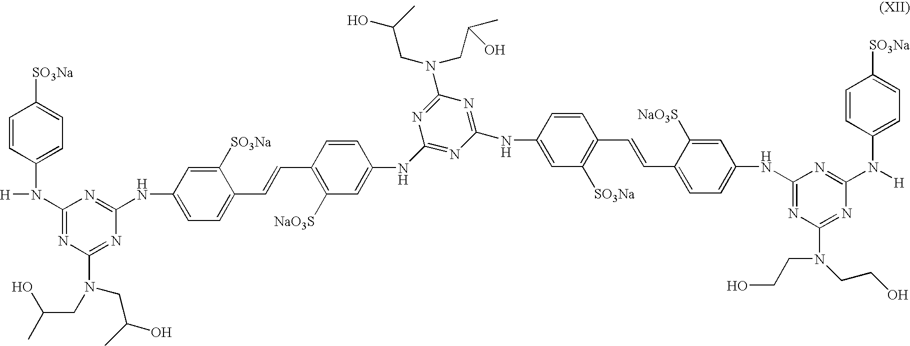

### Comparison Example 1

### Corresponds to Example 4 from EP-A-1 355 004

77.6 g of a membrane-filtered aqueous concentrate with an E1/1 value of 161 and a pH value of 8.5, which contains for the brightener of formula VII with R₁═R₃=a residue of the sodium salt of p-sulfanilic acid bonded through the nitrogen atom and R²═R⁴=a residue of diethanolamine bonded through the nitrogen atom, are mixed during agitation at room temperature with 22 g demineralized water and set to pH 9.0 with about 10% NaOH. A carrier-free brightener preparation is obtained with an E1/1 value of 125 in the form of a yellow-brown homogenous liquid. This corresponds to a brightener content of about 21%.

### Comparison Example 2

### Corresponds to Example 1 from EP-A-1 355 004

The procedure of that in example 1 is followed but the brightener of formula VII is used with R¹═R³=a residue of the sodium salt of p-sulfanilic acid bonded through the nitrogen atom and R²═R⁴=a residue of diisopropanolamine bonded through the nitrogen atom. A carrier-free brightener preparation is obtained with an E1/1 value of 125. This corresponds to a brightener content of about 23 wt %.

### APPLICATION EXAMPLES

### Application Example 1

A paper-coating mass is prepared from the following components:

379 parts chalk Hydrocarb 90
162 parts clay SPS
108.0 parts of a 50% styrene-butadiene latex
27 parts polyvinyl alcohol (20%) as cobinder
3.6 parts polysalt S (50%) as dispersant (basic polyacrylic acid, BASF AG)
320.7 parts water

### 5% NaOH.

The amount of NaOH is chosen so that a pH value of 8.8 results.

The coating mass is divided into 10 parts, and each part mixed with 0.2%, 0.4%, 0.8%, 1.2%, and 1.6 wt % of a brightener preparation from preparation example 4, concentrated to an E1/1 value of 125 (21 wt % brightener content with respect to the preparation), desalted by membrane filtration, and then agitated for 10 minutes. The added amounts refer to the white pigment content of the coating mass. For comparison, part of the coating mass is mixed in the same manner with the same amounts of brightener preparation as in comparison example 1. The brightened coating masses obtained are applied with a laboratory coating device (Erichsen Co., K-Control coater, model K202) onto wood-free paper with a basis weight of about 80 g/m². The coated papers are dried for 1 minute at 95° C. on a drying cylinder and then stored for 3 hours at 23° C. and 50% relative humidity. The coating-application weight was determined to be 15 g/m².

Measurement of the parameters L*, a*, and b* and determination of CIE whiteness were then performed with a whiteness measurement device (Datacolor Elrepho 2000).

The values obtained are shown in Tables 1 and 2.

TABLE 1
Brightener preparation from example 4 (E1/1 = 125).

| Amount (%) | CIE whiteness | L* | a* | b* |
|---|---|---|---|---|
| 0.2 | 99.37 | 94.60 | 0.86 | −2.79 |
| 0.4 | 104.12 | 94.67 | 1.11 | −3.81 |
| 0.8 | 107.5 | 94.85 | 1.01 | −4.37 |
| 1.2 | 107.09 | 94.96 | 0.63 | −4.33 |
| 1.6 | 103.41 | 95.05 | −0.15 | −3.47 |

TABLE 2
Brightener preparation from comparative example 1 (E1/1 = 125).

| Amount (%) | CIE whiteness | L* | a* | b* |
|---|---|---|---|---|
| 0.2 | 97.49 | 94.55 | 0.74 | −2.41 |
| 0.4 | 101.80 | 94.57 | 0.95 | −3.35 |
| 0.8 | 104.97 | 94.78 | 0.93 | −3.94 |
| 1.2 | 106.37 | 94.94 | 0.74 | −4.17 |
| 1.6 | 105.73 | 95.03 | 0.46 | −3.99 |

It can be seen that when the brightener according to the invention is used with the same E1/1 value in the PVA-containing coating color in the range of low use concentrations, better CIE whiteness values are obtained than from comparison example 1, which is not according to the invention.

### Application Example 2

We proceed as in application example 1, but use a brightener preparation from preparation example 1, concentrated, on the one hand, to an E1/1 value of 125 (≈23 wt % brightener with respect to the brightener preparation) and desalted by membrane filtration, and, on the other hand, the same amount of brightener preparation from comparison example 2.

The values obtained values are shown in Tables 3 and 4.

TABLE 3
Brightener preparation from example 1 (E1/1 = 125).

| Amount (%) | CIE whiteness | L* | a* | b* |
|---|---|---|---|---|
| 0.2 | 99.10 | 94.58 | 0.83 | −2.75 |
| 0.4 | 103.89 | 94.63 | 1.1 | −3.78 |
| 0.8 | 107.68 | 94.81 | 1.17 | −4.53 |
| 1.2 | 108.07 | 94.85 | 0.97 | −4.60 |
| 1.6 | 107.59 | 94.84 | 0.67 | −4.49 |

TABLE 4
Brightener preparation from comparison example 2 (E1/1 = 125).

| Amount (%) | CIE whiteness | L* | a* | b* |
|---|---|---|---|---|
| 0.2 | 97.66 | 94.58 | 0.77 | −2.43 |
| 0.4 | 101.92 | 94.63 | 0.98 | −3.34 |
| 0.8 | 106.47 | 94.74 | 1.12 | −4.30 |
| 1.2 | 108.72 | 94.75 | 1.09 | −4.79 |
| 1.6 | 109.65 | 94.99 | 0.90 | −4.88 |

It is apparent that with the same E1/1, use of the brightener according to the invention in the PVA-containing coating color in the range of low use concentrations leads to better CIE whitenesses than that from comparison example 2.

### Application Example 3

We proceed as in application example 1, but use, on the one hand, a preparation prepared by mixing from 5 parts by weight of the brightener preparation from preparation example 4 concentrated to an E1/1 value of 125 and desalted by membrane filtration and 95 parts by weight of the brightener preparation of comparison example 1, and, on the other hand, the same amount of brightener preparation from the comparison example.

The values obtained are shown in Tables 5 and 6.

TABLE 5
Preparation from 5 wt % desalted and membrane-filtered
brightener preparation from preparation example 4
(E1/1 = 125) and 95 wt % brightener preparation from
comparative example 1 (E1/1 = 125)/

| Amount (%) | CIE whiteness | L* | a* | b* |
|---|---|---|---|---|
| 0.2 | 97.30 | 94.49 | 0.72 | −2.39 |
| 0.4 | 101.98 | 94.65 | 0.94 | −3.35 |
| 0.8 | 106.61 | 94.81 | 1.04 | −4.29 |
| 1.2 | 107.28 | 94.85 | 0.83 | −4.47 |
| 1.6 | 107.83 | 94.88 | 0.63 | −4.53 |

TABLE 6
Brightener preparation from comparative example 1 (E1/1 = 125).

| Amount (%) | CIE whiteness | L* | a* | b* |
|---|---|---|---|---|
| 0.2 | 97.49 | 94.55 | 0.74 | −2.41 |
| 0.4 | 101.80 | 94.57 | 0.95 | −3.35 |
| 0.8 | 104.97 | 94.78 | 0.93 | −3.94 |
| 1.2 | 106.37 | 94.94 | 0.74 | −4.17 |
| 1.6 | 105.73 | 95.03 | 0.46 | −3.99 |

It is apparent that the preparation according to the invention has overall a better brightening effect than the brightener preparation not according to the invention.

### Application Example 4

The procedure of application example 1 is followed but, on the one hand, we use a preparation prepared by mixing from 5 parts by weight of a brightener preparation of preparation example concentrated to an E1/1 value of 125 and desalted by membrane filtration and 95 parts by weight of the brightener preparation of comparison example 1 and, on the other hand, the same amount of brightener preparation from comparison example 2.

The values obtained are shown in Tables 7 and 8.

TABLE 7
Preparation from 5 wt % desalted and membrane-filtered
brightener preparation from preparation example 1
(E1/1 = 125) and 95 wt % brightener preparation from
comparative example 2 (E1/1 = 125).

| Amount (%) | CIE whiteness | L* | a* | b* |
|---|---|---|---|---|
| 0.2 | 98.13 | 94.49 | 0.80 | −2.58 |
| 0.4 | 102.97 | 94.62 | 1.06 | −3.58 |
| 0.8 | 107.60 | 94.78 | 1.15 | −4.42 |
| 1.2 | 109.36 | 94.80 | 1.12 | −4.91 |
| 1.6 | 111.04 | 94.89 | 1.06 | −5.24 |

TABLE 8
Brightener preparation from comparison example 2 (E1/1 = 125).

| Amount (%) | CIE whiteness | L* | a* | b* |
|---|---|---|---|---|
| 0.2 | 97.66 | 94.58 | 0.77 | −2.43 |
| 0.4 | 101.92 | 94.63 | 0.98 | −3.34 |
| 0.8 | 106.47 | 94.74 | 1.12 | −4.30 |
| 1.2 | 108.72 | 94.75 | 1.09 | −4.79 |
| 1.6 | 109.65 | 94.99 | 0.90 | −4.88 |

It can be seen that the preparation according to the invention has overall a better brightening effect than the brightener preparation not according to the invention.

The preparations of the application examples also possess the same ratios as the corresponding weighed amount (95:5) after determination of the relevant surface percents of the brightener via the HPLC process as described in the description.

1. Compounds of formula I 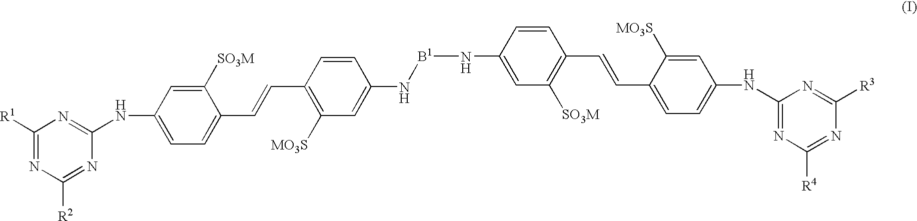
in which
R¹, R², R³, and R⁴ each, independently of one another, stand for OR⁵ or NR⁶R⁷, whereby
R⁵, R⁶, and R⁷ each, independently of one another, stand for hydrogen, substituted or unsubstituted alkyl, especially C₁-C₄ alkyl or substituted or unsubstituted aryl, especially C₆-C₁₀ aryl, whereby
R⁶, R⁷ can also form an aliphatic or aromatic ring together with the N atom to which they are bonded and optionally additional heteroatoms,
M stands for hydrogen, one equivalent of a mono- or divalent metal ion, especially from the group of alkali or alkaline-earth metals or an optionally organic substituted ammonium ion, and
B¹ stands for a bivalent bridge element.

2. Compounds according to claim 1, characterized in that at least one of the groups R¹ or R² has the same meaning as at least one of the groups R³ or R⁴.

3. Compounds according to claim 1, characterized in that R¹═R³ and R²═R⁴.

4. Compounds according to claim 1, characterized in that the groups R¹ to R⁴ are identical.

5. Compounds according to claim 1, characterized in that the bridge element B¹ preferably stands for a group of formula 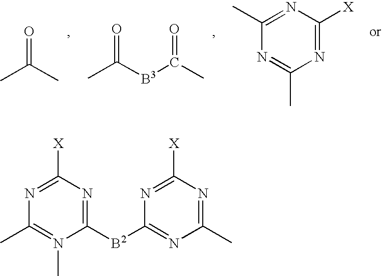
whereby
B³ denotes a bivalent, aliphatic, or aromatic group, especially 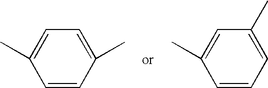
X stands for OR⁵ or NR⁶R⁷ and
R⁵, R⁶, and R⁷ each, independently of one another, has the meaning already mentioned and B² denotes a bridge element bonded through oxygen atoms or nitrogen atoms to the triazine residue, preferably an aliphatic bridge element.

6. Compounds according to claim 1, characterized in that the groups R¹, R², R³, and R⁴, independently of one another, stand for phenoxy, mono- or disulfonated phenoxy, phenylamino, mono- or disulfonated phenylamino, phenylamino substituted with C₁-C₃ alkyl, cyano, halogen, especially Cl or Br, COOR, CONH—R, NH—COR, SO₂NH—R, OR, also the groups morpholino, piperidino, pyrrolidino, —OC₁-C₄ alkyl, —NH—(C₁-C₄ alkyl), —N(C₁-C₄ alkyl)₂, —NH(C₂-C₄ alkylene)-OR, —N[(C₁-C₄-alkylene)-OR]₂, —NH(C₂-C₄ hydroxyalkyl), —N(C₂-C₄ hydroxyalkyl)₂, —NH(C₂-C₄ alkylene-O—C₂-C₄-alkylene-OR), an amino acid or amino acid salt of an amino-acid amide, from whose amino group a hydrogen atom is removed, —N(CH₃)(CH₂CH₂OH), —NH₂, OCH₂CH₂SO₃M, —NH—CH₂CH₂SO₃M, —N(CH₂CH₂SO₃M)₂ or —N(CH₂CH₂OH)CH₂CH₂CONH₂, in which R═H or C₁-C₃ alkyl and M has the above-mentioned meaning.

7. Compounds according to claim 1, characterized in that the groups R¹ to R⁴, independently of one another stand for —NH₂, —NH—CH₃, —NH—C₂H₅, —N(CH₃)₂, —N(C₂H₅)₂, —NH—C₂-C₄ hydroxyalkyl, especially —NH—CH₂CH₂OH, —N(C₂-C₄ hydroxyalkyl)₂, —NH—CH₂CH₂SO₃M, —NH—CH₂—CH₂—O—CH₂—CH₂—OH, —OCH₃, —OCH(CH₃)₂, —O—CH₂—CH₂—O—CH₃, —N(CH₂—CH₂—OH)₂, —N(CH₂—CHOH—CH₃)₂, morpholino, —N(CH₂—CH₂—OH)CH₂—CH₂—CONH₂, as well as groups of the formula 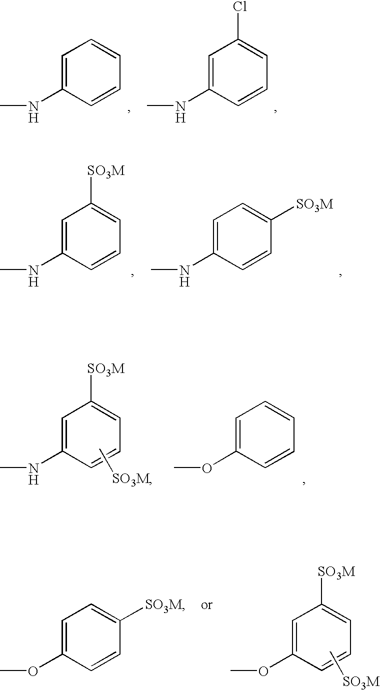
whereby
M has the above-mentioned meaning.

8. Compounds according to claim 1, characterized in that groups R¹ to R⁴, independently of each other, stand for 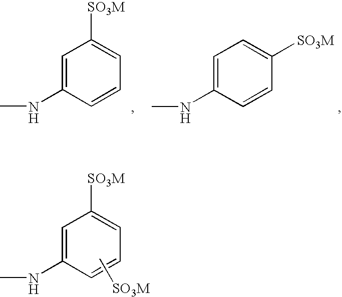
—NH—CH₂CH₂OH, —N(CH₂—CH₂—OH)₂, —N(CH₂—CHOH—CH₃)₂, aniline, or morpholino.

9. Compounds according to claim 1, characterized in that the compound of formula I corresponds to formula Ia 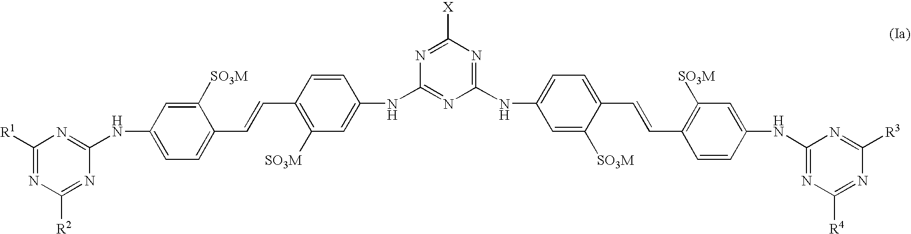
in which
R¹ and R³, independently of each other, stand for a group of the formula 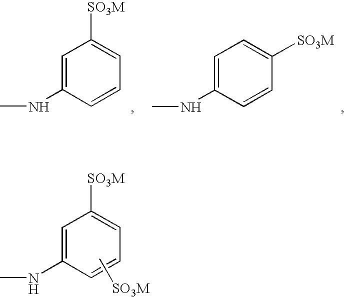
in which
M has the meaning according to claim 1,
R² and R⁴ independently of each, other stand for —NHCH₂—CH₂OH,
—N(CH₂CH₂OH)₂, 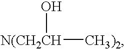
aniline, or morpholino and
X stands for OH or for —NHCH₂CH₂OH, —N(CH₂CH₂OH)₂, 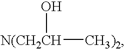
aniline, or morpholino.

10. A process for producing compounds according to claim 1, characterized in that a compound of formula II 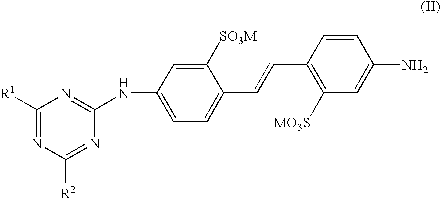
in which
R¹, R² and M having the meaning mentioned in at least one of claims 1 through 9, is converted with a compound of formula (III)
Y—B¹-Z (III)
in which
Y and Z independently of each other, stand for leaving groups that can be replaced by the free amino group of compounds of formula II and B¹ has the above-mentioned meaning, to a compound of formula 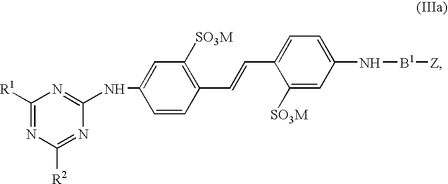
in which
R¹, R², B¹ and Z have the above-mentioned meaning and compound IIIa is further converted with a compound of formula IV 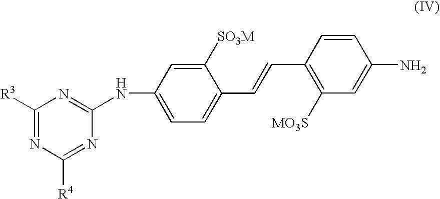
in which R³, R⁴ and M have the above-mentioned meaning, into a compound of formula I.

11. A preparation containing at least one compound of claim 1.

12. Preparations according to claim 11 containing, with respect to total brightener content, 1 to 15 area %, preferably 2 to 10, especially preferably 2.5 to 10, most especially preferably 3.0 to 10% of at least one compound according to at least one of the claims 1 through 9, in which the area % is measured in high-pressure liquid chromatogram at a wavelength of 350 nm on an aqueous measurement solution of the preparation, in which the amount of preparation expressed in grams weighed in per for 100 ml of measurement solution multiplied by its E1/1 value should be 50.

13. Preparations according to claim 11, characterized in that they contain water and 5 to 50 wt % total brightener.

14. Preparations according to claim 11, characterized in that a mixture of compound I according to claim 1 and compound VII is used as brightener 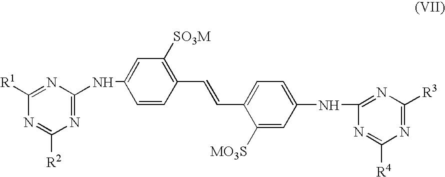
in which
R¹ to R⁴ and M independently of the meaning in formula I, have the meaning according to at least one of the claims 1 to 9.

15. Coating masses containing
water,
at least one white pigment,
at least one binder, especially a latex binder, and
at least one brightener according to claim 1 or a preparation according to claim 11.

16. Use of a compound according to claim 1 or a preparation or coating mass according to claim 11 to whiten of cellulose-containing materials, especially paper or cotton.
